(19) United States
(12) Patent Application Publication
(10) Pub. No.: US 2025/0344776 A1
(43) Pub. Date: Nov. 13, 2025

(54) MODIFIED BOXER BRIEF UNDERWEAR FOR MENSTRUAL CYCLES

(71) Applicant: Taylor Michelle Emery, Christiansburg, VA (US)

(72) Inventor: Taylor Michelle Emery, Christiansburg, VA (US)

(21) Appl. No.: 19/274,819

(22) Filed: Jul. 21, 2025

Related U.S. Application Data
(63) Continuation-in-part of application No. 18/301,217, filed on Apr. 15, 2023.

(51) Int. Cl. A41B 9/00; A41B 9/14; A41B 17/00

(52) U.S. Cl. CPC A41B 9/001; A41B 9/14; A41B 17/00

(57) ABSTRACT

Described herein are modified boxer brief underwear to accommodate for menstrual cycles. An extra piece of fabric is stitched longitudinally on both ends within the pelvic area for the purpose of application of pads with wings and panty liners. Due to the nature of the material used, menstrual products are able to stick easily and are therefore held in place during use.

### TECHNICAL FIELD

The subject matter disclosed herein is generally directed to modified boxer brief underwear designed to accommodate menstrual cycles. An extra piece of fabric is stitched longitudinally on both ends within the pelvic area for the purpose of application of pads with wings and panty liners. Due to the nature of the material used, menstrual products are able to stick easily and are therefore held in place during use.

### BACKGROUND

Designing boxer briefs that accommodate menstrual products is a significant step forward in addressing the diverse needs and preferences of individuals who menstruate. Traditionally, the focus of underwear designed for accommodating menstruation products has been primarily for women's panties and thongs, with no consideration given to menstruation-specific boxer briefs that are typically designed for men. However, as awareness grows about the importance of inclusive design in feminine hygiene products, there is a recognized need for boxer briefs that can effectively accommodate menstrual products such as pads and panty liners.

One of the key reasons for the importance of designing boxer briefs with menstrual product accommodation in mind is inclusivity. Menstruation is a natural biological process that affects individuals across the gender spectrum, including transgender men and non-binary individuals. These individuals may prefer boxer briefs for their comfort and style but still require menstrual products for managing their periods. By creating boxer briefs that can accommodate menstrual products, designers can ensure that individuals of all genders have access to underwear that meets their specific needs during menstruation.

There is a notable gap in the market for boxer briefs specifically designed to accommodate pads and panty liners. While many styles of underwear that accommodate menstruation exist, such as bikini briefs or boy shorts, they may not always provide the ideal fit or coverage for individuals who prefer to wear boxer briefs or want additional protection during menstruation. This gap underscores the need for a modified boxer brief to accommodate menstrual cycles, a versatile undergarment that combines the functionality of traditional men's underwear with the convenience and comfort of boxer briefs. By offering a longer leg length and a snug, supportive fit, menstrual boxer briefs provide ample coverage for pads and panty liners, helping to prevent leaks and minimize discomfort.

Another important aspect is comfort and functionality. Traditional underwear may not always provide the necessary coverage or support for securely holding menstrual products in place. This can lead to discomfort, leaks, and a lack of confidence during menstruation. Boxer briefs designed with additional fabric layers that can provide the support and security needed to keep menstrual products in place, reducing the risk of leaks and increasing comfort.

In conclusion, there is an understood need for designing boxer briefs that accommodate menstrual products. By considering the diverse needs and preferences of individuals who menstruate, underwear can be designed that supports their well-being and empowers them to navigate menstruation with case and confidence. The development of menstrual boxer briefs addresses a crucial need within the realm of feminine hygiene, providing a practical and inclusive solution for individuals during menstruation.

Citation or identification of any document in this application is not an admission that such a document is available as prior art to the present disclosure.

### SUMMARY

The above objectives are accomplished according to the present disclosure by providing a modified boxer brief underwear designed to accommodate menstrual cycles. In the first embodiment, the modified boxer brief for menstrual cycles comprises a waistband at top of the boxer brief and a body. The body further comprises a right leg portion, a right leg opening, a left leg portion, and a left leg opening. The right leg portion is configured with at least one right leg seam and the left leg portion is configured with at least one left leg seam. The body further comprises a pelvic area in between the right leg portion and the left leg portion, an anterior arched seam configured to span from the right leg portion to the left leg portion, a posterior arched seam configured to span from the right leg portion to the left leg portion, and a longitudinal scam configured to span from the arched seam to the waistband. The pelvic area is configured to include an additional piece of fabric on the interior. Further, the fabric used is cotton, nylon, rayon, spandex, polyester, silk, or modal. Again further, the fabric is configured to adhere to menstrual products such as pads and panty liners. Still further, the additional piece of fabric on the interior pelvic area is configured to hold menstrual products such as pads and panty liners. Still yet further, the arched seam is configured on the front and the back body to hold the additional piece of fabric in place. Still again, the additional piece of fabric is configured to be stitched on the left and the right side by the longitudinal seam. Further still, the longitudinal seam is configured from the left side and the right side of the additional piece of fabric to the waistband to further secure the additional piece of fabric. Still yet, the waistband is configured to secure the modified boxer brief onto the waist. Still further yet, the right leg opening and the left leg opening allows for breathability of the pelvic area.

In a further embodiment, a modified boxer brief for menstrual cycles is formed. The modified boxer brief for menstrual cycles forms a waistband at top of the boxer brief and a body. The body further forms a right leg portion, a right leg opening, a left leg portion, and a left leg opening. The right leg portion is configured with at least one right leg seam and the left leg portion is configured with at least one left leg seam. The body further forms a pelvic area in between the right leg portion and the left leg portion, an anterior arched seam configured to span from the right leg portion to the left leg portion, a posterior arched seam configured to span from the right leg portion to the left leg portion, and a longitudinal seam configured to span from the arched seam to the waistband. The pelvic area is configured to include an additional piece of fabric on the interior. Further, the fabric used is cotton, nylon, rayon, spandex, polyester, silk, or modal. Again further, the fabric is configured to adhere to menstrual products such as pads and panty liners. Still further, the additional piece of fabric on the interior pelvic area is configured to hold menstrual products such as pads and panty liners. Still yet further, the arched seam is configured on the front and the back body to hold the additional piece of fabric in place. Still again, the additional piece of fabric is configured to be stitched on the left and the right side by the longitudinal seam. Further still, the longitudinal seam is configured from the left side and the right side of the additional piece of fabric to the waistband to further secure the additional piece of fabric. Still yet, the waistband is configured to secure the modified boxer brief onto the waist. Still further yet, the right leg opening and the left leg opening allows for breathability of the pelvic area.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows the front of the boxer briefs.

FIG. 2 shows the fabric lining on the pelvic area of the interior boxer briefs.

FIG. 3 shows the back of the boxer briefs.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g., given data set, art accepted standard, and/or with e.g., a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic.

The terms “subject,” “individual,” and “patient” are used interchangeably herein to refer to a vertebrate, preferably a mammal, more preferably a human.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the modified boxer brief underwear described herein can be presented as a combination kit. As used herein, the terms “combination kit” or “kit of parts” refers to the compounds, compositions, embodiments and any additional components that are used to package, sell, market, deliver, and/or assemble the combination of elements or a single element, such as the modified boxer brief underwear, contained therein. Such additional components include, but are not limited to, packaging, syringes, blister packages, wrapping, and the like. When one or more of the compounds, compositions, embodiments, described herein or a combination thereof (e.g., modified boxer brief underwear contained in the kit are provided simultaneously, the combination kit can contain the modified boxer brief underwear in a single embodiment, such as a single piece unit or in separate combinations, such as a modified boxer brief underwear accompanied by a menstrual pad, etc., that may be added to or used in conjunction with the modified boxer brief underwear.) or in separate embodiments. When the compounds, compositions, embodiments described herein or a combination thereof and/or kit components are not assembled simultaneously, the combination kit can contain each component in separate embodiments. The separate kit components can be contained in a single package or in separate packages within the kit.

In some embodiments, the combination kit also includes instructions printed on or otherwise contained in a tangible medium of expression. The instructions can provide information regarding the content of the compounds and/or embodiments, safety information regarding the content of the compounds and embodiments, indications for use, and/or recommended care for the compound(s) and/or embodiments contained therein. In some embodiments, the instructions can provide directions and instructions for assembling the compounds and/or embodiments described herein to a subject in need thereof. In some embodiments, the instructions can provide one or more embodiments of the methods for using the modified boxer brief underwear such as any of the methods described in greater detail elsewhere herein.

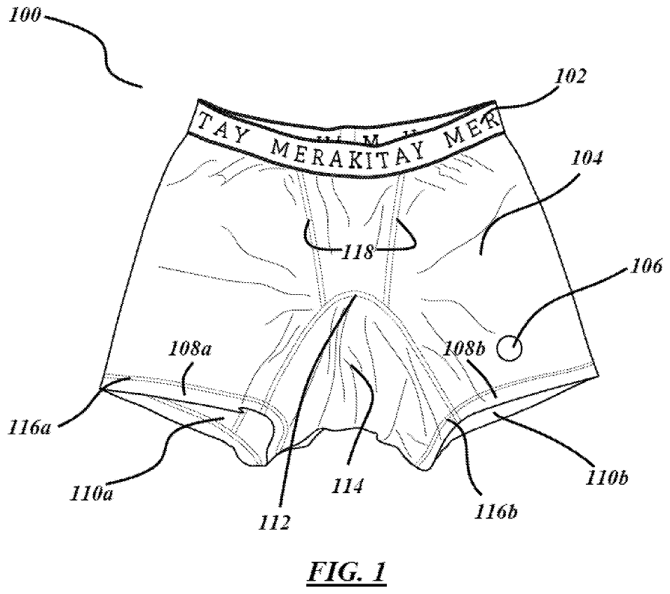

The current disclosure provides modified boxer brief underwear 100. FIGS. 1, 2 and 3 are illustrative embodiments of the modified boxer brief underwear 100. Modified boxer brief underwear 100 may be shaped as a square, rectangle, or other polygon and may be fabricated from various materials including, without limitation, cotton, nylon, rayon, spandex, polyester, silk, or modal. The type of fabric used for modified boxer brief underwear 100 is intended to be accommodate menstruation products such as pads and panty liners by allowing them to adhere to the fabric. Modified boxer brief underwear 100 comes in a variety of sizes ranging from XXS to XXXL. Modified boxer brief underwear 100 may range in length from 8 inches to 20 inches and width from 11 inches to 35 inches depending on the size of the modified boxer brief underwear. The inseam of modified boxer brief underwear 100 may range from 3 inches to 9 inches.

FIG. 1 provides modified boxer brief underwear 100 from the front view. The modified boxer brief 100 includes body 104 with waistband 102 at the top of the body and right leg portion 108a and left leg portion 108b at the bottom of the body. Body 104 may also include embroidered area 106 with the company's brand name inside. This embroidered area may be shaped as a circle, square, rectangle, or other polygon. The right leg portion 108a is surrounded by right leg scam 116a and leads to right leg opening 110a. The left leg portion 108b is surrounded by left leg scam 116b and leads to left leg opening 110b. In between the right leg portion 108a and left leg portion 108b is pelvic area 114. Pelvic area 114 is enclosed on the front portion of modified boxer brief 100 by arched seam 112. Anterior arched seam 112 spans from right leg portion 108a to left leg portion 108b. Longitudinal seam 118 is configured to span from the at least one arched seam to the at least one waistband.

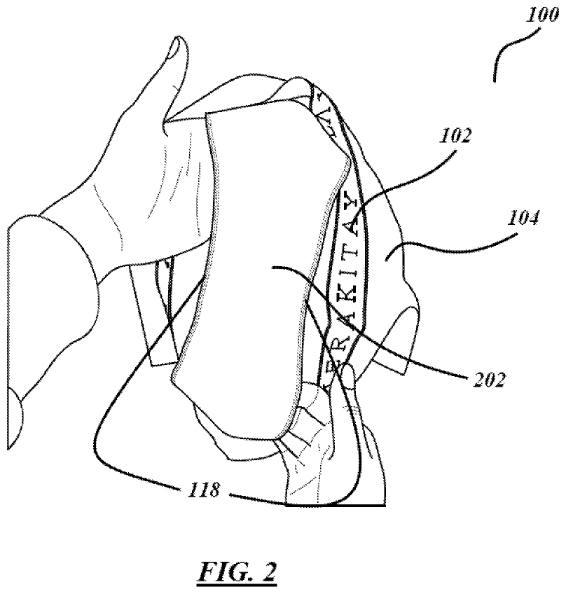

FIG. 2 provides modified boxer brief underwear 100 with a view of the fabric lining on the pelvic area of the interior boxer briefs. Pelvic area 114 is configured to include an additional piece of fabric 202 on the interior to accommodate menstruation products and allow them to adhere to the surface of the additional piece of fabric 202. The range of the additional piece of fabric 202 is 4 inches to 14 inches in length. The additional piece of fabric 202 is held into place by longitudinal seam 118. This longitudinal scam 118 extends from each side of the additional piece of fabric 202 up to the waistband 102 to further secure the additional piece of fabric 202 in place. The additional piece of fabric 202 is also secured into place by anterior arched scam 112 and posterior arched seam 302. Body 104 and waistband 102 are also visible in FIG. 2.

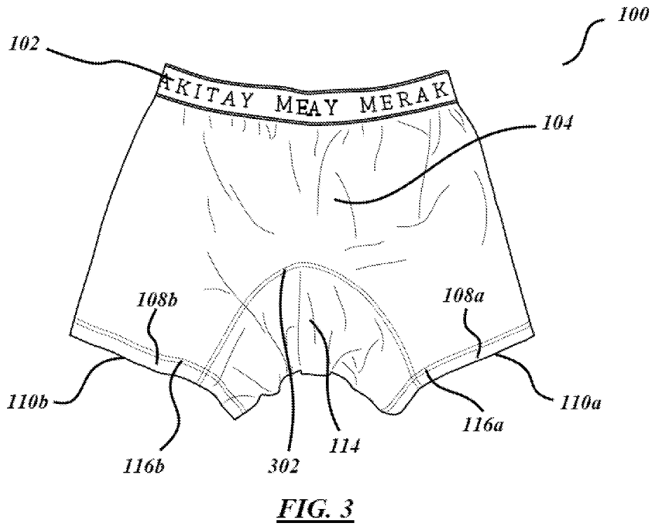

FIG. 3 provides modified boxer brief underwear 100 from the back view. Body 104 is visible and enclosed by waistband 102 on the top and right leg portion 108a and left leg portion 108b on the bottom. The right leg portion 108a is surrounded by right leg seam 116a and leads to right leg opening 110a. The left leg portion 108b is surrounded by left leg seam 116b and leads to left leg opening 110b. In between the right leg portion 108a and left leg portion 108b is pelvic area 114. Posterior arched scam 302 spans from right leg portion 108a to left leg portion 108b.

The modified boxer brief underwear may be integrated into yoga pants, biker shorts, and spandex for athletes.

Meraki in Greek means doing something with soul, creativity, and love. The idea of putting something of oneself into everything one does. MerakiTay is a unisex clothing brand started because of a love for fashion. Each collection presented in MerakiTay has a specific meaning. They represent things that everyone should incorporate into their mindsets, or something they've been through, or just something to relate to. Taylor Emery, former professional basketball player, former Player-Development coach, and now veterinarian, established MerakiTay in April of 2021. A member of the LGBTQ community, and a woman who feels more comfortable wearing more masculine clothes, Taylor founded MerakiTay with the idea of producing clothing for everyone of all genders.

Various modifications and variations of the described methods, compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A modified boxer brief for menstrual cycles comprising:
at least one waistband at top of the boxer brief;
at least one body;
wherein the at least one body further comprises;
at least one right leg portion;
wherein the at least one right leg portion is configured with at least one right leg seam;
at least one right leg opening;
at least one left leg portion;
wherein the at least one left leg portion is configured with at least one left leg seam;
at least one left leg opening;
at least one pelvic area in between the at least one right leg portion and the at least on left leg portion;
wherein the at least one pelvic area is configured to include at least one additional piece of fabric on the interior;
at least one anterior arched seam configured to span from the at least one right leg portion to the at least one left leg portion;
at least one posterior arched seam configured to span from the at least one right leg portion to the at least one left leg portion; and
at least one longitudinal seam configured to span from the at least one arched seam to the at least one waistband.

2. The modified boxer brief for menstrual cycles of claim 1, wherein the fabric used is cotton, nylon, rayon, spandex, polyester, silk, or modal.

3. The modified boxer brief for menstrual cycles of claim 2, wherein the fabric is configured to adhere to menstrual products such as pads and panty liners.

4. The modified boxer brief for menstrual cycles of claim 1, wherein the at least one additional piece of fabric on the at least one interior pelvic area is configured to hold menstrual products such as pads and panty liners.

5. The modified boxer brief for menstrual cycles of claim 4, wherein the at least one arched seam is configured on the front and the back body to hold the at least one additional piece of fabric in place.

6. The modified boxer brief for menstrual cycles of claim 4, wherein the at least one additional piece of fabric is configured to be stitched on the left and the right side by the at least one longitudinal seam.

7. The modified boxer brief for menstrual cycles of claim 6, wherein at least one longitudinal seam is configured from the left side and the right side of the at least one additional piece of fabric to the at least one waistband to further secure the at least one additional piece of fabric.

8. The modified boxer brief for menstrual cycles of claim 1, wherein the at least one waistband is configured to secure the at least one modified boxer brief onto the waist.

9. The modified boxer brief for menstrual cycles of claim 1, wherein the at least one right leg opening and the at least one left leg opening allows for breathability of the at least one pelvic area.

10. A modified boxer brief for menstrual cycles comprising:
forming at least one waistband at top of the boxer brief;
forming at least one body;
wherein the at least one body further comprises;
forming at least one right leg portion;
wherein the at least one right leg portion is configured with at least one right leg seam;
forming at least one right leg opening;
forming at least one left leg portion;
wherein the at least one left leg portion is configured with at least one left leg seam;
forming at least one left leg opening;
forming at least one pelvic area in between the at least one right leg portion and the at least on left leg portion;
wherein the at least one pelvic area is configured to include at least one additional piece of fabric on the interior;
forming at least one anterior arched seam configured to span from the at least one right leg portion to the at least one left leg portion;
forming at least one posterior arched seam configured to span from the at least one right leg portion to the at least one left leg portion; and
forming at least one longitudinal seam configured to span from the at least one arched seam to the at least one waistband.

11. The modified boxer brief for menstrual cycles of claim 10, further comprising forming the fabric used is cotton, nylon, rayon, spandex, polyester, silk, or modal.

12. The modified boxer brief for menstrual cycles of claim 11, further comprising forming the fabric is configured to adhere to menstrual products such as pads and panty liners.

13. The modified boxer brief for menstrual cycles of claim 10, further comprising forming the at least one additional piece of fabric on the at least one interior pelvic area is configured to hold menstrual products such as pads and panty liners.

14. The modified boxer brief for menstrual cycles of claim 13, further comprising forming the at least one arched seam is configured on the front and the back body to hold the at least one additional piece of fabric in place.

15. The modified boxer brief for menstrual cycles of claim 13, wherein the at least one additional piece of fabric is configured to be stitched on the left and the right side by the at least one longitudinal seam.

16. The modified boxer brief for menstrual cycles of claim 15, wherein at least one longitudinal seam is configured from the left side and the right side of the at least one additional piece of fabric to the at least one waistband to further secure the at least one additional piece of fabric.

17. The modified boxer brief for menstrual cycles of claim 10, further comprising forming the at least one waistband is configured to secure the at least one modified boxer brief onto the waist.

18. The modified boxer brief for menstrual cycles of claim 10, further comprising forming the at least one right leg opening and the at least one left leg opening allows for breathability of the pelvic area.
